(19) United States
(12) Patent Application Publication
(10) Pub. No.: US 2026/0272423 A1
(43) Pub. Date: Sep. 17, 2026

(54) METHOD FOR PROVIDING INFORMATION FOR THE PREDICTION OF LEFT VENTRICULAR OUTFLOW TRACT OBSTRUCTION IN HYPERTROPHIC CARDIOMYOPATHY

(71) Applicant: ONTACT HEALTH CO., LTD., Seoul (KR)

(72) Inventors: Seung Ah Lee, Seoul (KR); Jae Ik Jeon, Gyeonggi-do (KR); Ji Yeon Kim, Seoul (KR)

(73) Assignee: ONTACT HEALTH CO., LTD., Seoul (KR)

(21) Appl. No.: 19/376,521

(22) Filed: Oct. 31, 2025

(30) Foreign Application Priority Data
Mar. 11, 2025 (KR) 1020250031713
Oct. 30, 2025 (KR) 1020250160865

(51) Int. Cl. A61B 8/00; A61B 8/08; G06T 5/70; G06T 7/246

(52) U.S. Cl. CPC A61B 8/5223; A61B 8/0883; A61B 8/5269; G06T 5/70; G06T 7/246; G06T 2207/10132; G06T 2207/20084; G06T 2207/30048

(57) ABSTRACT

The present disclosure relates to method for providing information for the prediction of left ventricular outflow tract obstruction in hypertrophic cardiomyopathy, in particular, the method can comprise obtaining an echocardiographic image of the patient and performing a preprocessing, inputting a preprocessed echocardiographic image into a pre-trained artificial intelligence model to extract spatial structural features and temporal motion features of the myocardium, either individually or in combination, from a single echocardiographic image as input, and to predict whether there is left ventricular outflow tract obstruction based on the extracted features and output a prediction result and generating, when a prediction result based on the preprocessed echocardiographic image is output from the artificial intelligence model, prognostic assessment information for the patient on the basis of the prediction result.

### CLAIM FOR PRIORITY

This application claims priority to Korean Patent Application No. 10-2025-0031713, filed on Mar. 11, 2025, and to Korean Patent Application No. 10-2025-0160865, filed on Oct. 30, 2025, both of which are incorporated by reference in entirety for all purposes. The USPTO is invited to retrieve the priority documents using the provided DAS codes.

### BACKGROUND

### Field

The present disclosure relates to a method for providing information for the prediction of left ventricular outflow tract obstruction in hypertrophic cardiomyopathy.

### Description of the Related Art

Hypertrophic cardiomyopathy (HCM) is a relatively common hereditary myocardial disease worldwide, and it is reported that it occurs in about 1 in 200 to 500 people. HCM clinically presents with a variety of symptoms, ranging from asymptomatic to dyspnea, chest pain, and fainting, and in some patients, it also acts as a major cause of sudden cardiac death. In particular, the presence and extent of left ventricular outflow tract obstruction (LVOTO) is known as a key factor in determining the patient's symptoms and prognosis.

Currently, assessment at clinical sites is mainly performed using transthoracic echocardiography (TTE), and provocation tests such as multiple transthoracic echocardiographic views, Doppler tests, and Valsalva are performed together. However, these assessments require a high degree of expertise, the accuracy can vary depending on the patient's cooperation and test environment, and their application is limited in community hospitals or emergency situations.

In addition, although echocardiographic analysis techniques using artificial intelligence have been proposed in existing studies, most of them were focused on specific diseases such as aortic valve stenosis and diastolic function assessment, and a technique that precisely reflects dynamic characteristics of left ventricular outflow tract obstruction, which is important for predicting the prognosis of patients with hypertrophic cardiomyopathy, in a single image has not been sufficiently presented.

Therefore, there is a steady demand for assistive technique that can be used immediately in real clinical settings and automates the prediction and prognosis of left ventricular outflow tract obstruction, which is important in clinical management of patients with hypertrophic cardiomyopathy.

The background technology of the present disclosure was written to facilitate understanding of the present disclosure. It should not be understood that the matters described in the background technology of the invention exist as prior art.

### BRIEF DESCRIPTION OF DRAWINGS

FIG. 1 is a schematic diagram for explaining a system for providing information for the prediction of left ventricular outflow tract obstruction in hypertrophic cardiomyopathy, according to an embodiment of the present disclosure.

FIG. 2 is a block diagram showing the configuration of a service server that performs the task of providing information for the prediction of left ventricular outflow tract obstruction in hypertrophic cardiomyopathy, according to an embodiment of the present disclosure.

FIG. 3 is a block diagram illustrating a configuration of a user terminal receiving prognostic assessment information, according to an embodiment of the present disclosure.

FIG. 4 is a flowchart schematically illustrating a method for providing information for the prediction of left ventricular outflow tract obstruction in hypertrophic cardiomyopathy, according to an embodiment of the present disclosure.

FIGS. 5A and 5B are diagrams schematically illustrating a pipeline for predicting whether there is left ventricular outflow tract obstruction based on a single echocardiographic image using an artificial intelligence model, according to some embodiments of the present disclosure.

FIGS. 6A to 6C are diagrams schematically illustrating a process of training and assessing an artificial intelligence model for predicting left ventricular outflow tract obstruction in hypertrophic cardiomyopathy, according to some embodiments of the present disclosure.

FIGS. 7A and 7B are diagrams illustrating examples of application of an M-mode generation process and a sampling guide based on a prediction point in an artificial intelligence model according to some embodiments of the present disclosure.

FIG. 8 is a diagram illustrating the structure of an artificial intelligence model for each M-mode sampling scheme according to an embodiment of the present disclosure.

FIGS. 9A and 9B are diagrams illustrating performance assessment results using internal and external validation datasets of an artificial intelligence model, according to some embodiments of the present disclosure.

FIG. 10 is a diagram showing a visual comparison result according to whether there is left ventricular outflow tract obstruction predicted by an artificial intelligence model, according to some embodiments of the present disclosure.

FIG. 11 is a diagram illustrating an example of interpretation of an M-mode image generated by an artificial intelligence model as a prediction point-based sampling guide, according to an embodiment of the present disclosure.

FIG. 12 is a view illustrating an example of tracking Mavacamten treatment response to patients with obstructive hypertrophic cardiomyopathy using an artificial intelligence model, according to an embodiment of the present disclosure.

### SUMMARY

The prognosis of patients with hypertrophic cardiomyopathy varies greatly depending on the presence and severity of left ventricular outflow tract obstruction. However, the existing diagnosis relies on multiple echocardiographic images and Doppler measurements, making it difficult to apply in low-skilled examiners or resource-limited environments, and there is a problem of inconsistent prognosis judgments.

In addition, existing artificial intelligence-based echocardiography analysis studies were focused on specific disease classification or single index analysis rather than single image-based prognosis prediction, which could be easily secured, so there was a limit to the clinical prognosis prediction of hypertrophic cardiomyopathy patients. As a result, screening patients at early risk and monitoring of treatment responses were limited, and patient-specific prognosis management was also limited.

Therefore, there is a need for a new technical means to automatically predict whether there is left ventricular outflow tract obstruction and the patient's prognosis accordingly using a single echocardiographic image of patients with hypertrophic cardiomyopathy. In particular, there is a need for a system that can precisely analyze spatial and temporal myocardial movement characteristics through an artificial intelligence model and use it for patient-specific prognostic assessment and treatment response tracking.

Accordingly, the inventors of the present disclosure quantitatively calculate the possibility of left ventricular outflow tract obstruction with a single echocardiographic image as an input, predict the clinical prognosis of patients with hypertrophic cardiomyopathy based on the results, and further recognize the need for an artificial intelligence-based prognostic assessment framework that can reflect changes before and after treatment.

Therefore, an object to be achieved by the present disclosure is to provide a method for providing information for the prediction of left ventricular outflow tract obstruction in hypertrophic cardiomyopathy, by obtaining an echocardiographic image of patients with hypertrophic cardiomyopathy and analyzing the echocardiographic image with an artificial intelligence model, to assess whether there is left ventricular outflow tract obstruction and the prognosis of the patient, and to utilize the results in patient management and treatment decisions.

Objects of the present disclosure are not limited to the above-mentioned objects, and other objects, which are not mentioned above, can be clearly understood by those skilled in the art from the following descriptions.

In order to solve the above-described problems, a method for providing information for the prediction of left ventricular outflow tract obstruction in hypertrophic cardiomyopathy according to an embodiment of the present disclosure is provided. The method can comprise obtaining an echocardiographic image of the patient and performing a preprocessing; inputting a preprocessed echocardiographic image into a pre-trained artificial intelligence model to extract spatial structural features and temporal motion features of the myocardium, either individually or in combination, from a single echocardiographic image as input, and to predict whether there is left ventricular outflow tract obstruction based on the extracted features and output a prediction result; and generating, when a prediction result based on the preprocessed echocardiographic image is output from the artificial intelligence model, prognostic assessment information of the patient on the basis of the prediction result.

According to a feature of the present disclosure, performing the preprocessing can comprise performing at least one of noise removal, luminance correction, contrast correction, image resolution normalization, region of interest (ROI) extraction, time interval division, or frame-to-frame alignment in the echocardiographic image.

According to a feature of the present disclosure, the artificial intelligence model can comprise a first neural network for extracting spatial structural features from the preprocessed echocardiographic image and a second neural network for extracting temporal motion features from a frame sequence of the preprocessed echocardiographic image.

According to a feature of the present disclosure, the first neural network is implemented as a 3D convolutional neural network (3D-CNN), and the second neural network can be a 2D convolutional neural network (2D-CNN) or a transformer-based network configured to analyze a temporal changes in myocardial motion using an automatically generated M-mode representation.

According to a feature of the present disclosure, the second neural network can generate a multi-level M-mode feature map by automatically tracking a myocardial motion trajectory in the echocardiographic image, and embed the multi-level M-mode feature map to improve the accuracy of prediction of left ventricular outflow tract obstruction.

According to a feature of the present disclosure, the artificial intelligence model can predict whether there is left ventricular outflow tract obstruction by combining the embedding result output from the second neural network with the output of the first neural network.

According to a feature of the present disclosure, the prediction result can be a binary classification result for classifying the presence of left ventricular outflow tract obstruction, or can include at least one of continuous value (score) quantitatively predicted for the pressure gradient of the left ventricular outflow tract.

According to a feature of the present disclosure, the prediction result can be calculated as a prediction index (DLI-LVOTO) obtained by quantifying the degree of left ventricular outflow tract obstruction, and the prediction index can be calculated based on at least one of a contraction speed of a myocardium, a movement distance, or a timing difference in the preprocessed echocardiographic images.

According to a feature of the present disclosure, generating the prognostic assessment information can include combining the prediction result with at least one piece of clinical information (age, sex, heart rate, drug administration histories, etc.) about the patient to calculate a prognosis grade or risk level, and outputting a treatment response prediction result according to the calculated prognosis grade or risk level.

In order to solve the above-described problems, an apparatus for providing information for the prediction of left ventricular outflow tract obstruction in hypertrophic cardiomyopathy according to an embodiment of the present disclosure is provided. the apparatus comprises a communication interface; a memory; and a processor operatively connected with the communication interface and the memory, the processor being configured to obtain an echocardiographic image of a patient and perform pre-processing, extract spatial structural features and temporal motion features of the myocardium, either individually or in combination, using a single echocardiographic image as an input, to input the pre-processed echocardiographic image to an artificial intelligence model that is pre-trained to predict whether there is left ventricular outflow tract obstruction on the basis of the extracted features and to output a prediction result, and generate prognostic assessment information for the patient on the basis of the prediction result, when a prediction result based on the pre-processed echocardiographic image is output from the artificial intelligence model.

Other detailed matters of the embodiments are included in the description and drawings of the present disclosure.

The present disclosure assesses whether there is left ventricular outflow tract obstruction and the prognosis by acquiring echocardiographic images of patients with hypertrophic cardiomyopathy and analyzing them with an artificial intelligence model, and allows the results to be utilized in patient management and treatment decisions.

The effects according to the present disclosure are not limited to the contents exemplified above, and more various effects are included in the present disclosure.

### DETAILED DESCRIPTION OF THE EMBODIMENT

The advantages and features of the embodiments of the present disclosure, and the methods for achieving them, will become clear with reference to the embodiments described in detail below together with the attached drawings. However, the present disclosure is not limited to the embodiments disclosed below, but can be implemented in various different forms, and these embodiments are provided only to make the disclosure of the present disclosure complete and to fully inform a person having ordinary knowledge in the technical field to which the present disclosure belongs of the scope of the disclosure, and the present disclosure is defined only by the scope of the claims. In relation to the description of the drawings, similar reference numerals can be used for similar components.

In the present disclosure, the expressions “have”, “can have”, “include”, or “can include” refer to the presence of a feature (for example, a numerical value, function, operation, or component such as a part) and do not exclude the presence of additional features.

In the present disclosure, the expressions “A or B”, “at least one of A or/and B”, or “one or more of A or/and B” can include all possible combinations of the items listed together. For example, “A or B”, “at least one of A and B”, or “at least one of A or B” can all refer to (1) including at least one A, (2) including at least one B, or (3) including both at least one A and at least one B.

The expressions “first”, “second”, “firstly”, or “secondly”, used in the present disclosure can describe various components, regardless of order and/or importance, and are used only to distinguish one component from another, but do not limit the components. For example, a first user device and a second user device can represent different user devices, regardless of order or importance. For example, without departing from the scope of the rights set forth in the present disclosure, a first component can be referred to as a second component, and similarly, a second component can be referred to as a first component.

When a component (for example, a first component) is referred to as being “(operatively or communicatively) coupled with/to” or “connected to” another component (for example, a second component), it should be understood that said component can be directly connected to said other component, or can be connected via another component (for example, a third component). Meanwhile, when it is said that a component (for example, a first component) is “directly connected” or “directly connected” to another component (for example, a second component), it can be understood that no other component (for example, a third component) exists between said component and said other component.

The expression “configured to” as used in the present disclosure can be used interchangeably with, for example, “suitable for”, “having the capacity to”, “designed to”, “adapted to”, “made to”, or “capable of, depending on the situation. “The term “configured (or set) to” does not necessarily mean that something is “specifically designed to” in terms of hardware. Instead, in some contexts, the expression “an apparatus configured to” can mean that the apparatus is “capable of” doing something in conjunction with other apparatus or components. For example, the phrase “a processor configured (or set) to perform A, B, and C” can mean a dedicated processor (for example, an embedded processor) for performing the operations, or a general-purpose processor (for example, a CPU or an application processor) that can perform the operations by executing one or more software programs stored in a memory device.

The terms used in the present disclosure are used only to describe specific embodiments and cannot be intended to limit the scope of other embodiments. The singular expression can include the plural expression unless the context clearly indicates otherwise. Terms used herein, including technical or scientific terms, can have the same meaning as commonly understood by a person of ordinary skill in the art described in the present disclosure. Terms defined in general dictionaries among the terms used in the present disclosure can be interpreted as having the same or similar meaning as they have in the context of the relevant technology, and shall not be interpreted in an ideal or excessively formal sense unless explicitly defined in the present disclosure. In some cases, even if a term is defined in the present disclosure, it cannot be interpreted to exclude embodiments of the present disclosure.

Each feature of the various embodiments of the present disclosure can be partially or entirely combined or combined with each other, and as can be fully understood by those skilled in the art, various technical connections and operations are possible, and each embodiment may be implemented independently of each other or may be implemented together in a related relationship.

For clarity in the interpretation of this specification, the terms used in this specification are defined below.

Hereinafter, a device referred to as a “service server” according to the present disclosure can mean one physically independent server, but is not limited thereto, and may be a single virtual machine, and is intended to cover all one module or program, or docker operating in one virtual or physical machine.

Hereinafter, preferred embodiments of the present disclosure will be described in detail with reference to the accompanying drawings.

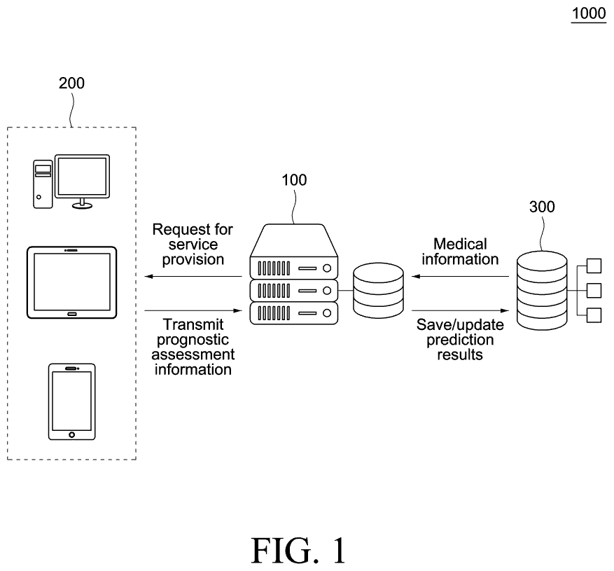

FIG. 1 is a schematic diagram for explaining a system for providing information for the prediction of left ventricular outflow tract obstruction in hypertrophic cardiomyopathy according to an embodiment of the present disclosure.

Referring to FIG. 1, a system 1000 for providing information for the prediction of left ventricular outflow tract obstruction in hypertrophic cardiomyopathy (hereinafter, referred to as ‘information provision system’) according to an embodiment of the present disclosure can be a system configured to automatically predict whether there is left ventricular outflow tract obstruction (LVOTO), and the prognosis of the patient thereby, and provide prognostic assessment information based on the assessment results. Accordingly, the information provision system 1000 can include a service server 100, a user terminal 200, and a medical information providing apparatus 300.

Service server 100 can correspond to a web server or a cloud server type, and refers to a device that receives and preprocesses echocardiographic images, analyzes them using an artificial intelligence model, calculates prediction results, and generates prognostic assessment information in information provision system 1000 of the present disclosure. Service server 100 can store and manage prediction results, prognostic assessment information, prediction indicators (e.g., DLI-LVOTO) obtained during the analysis process in a database, and can transmit and receive various data in real time in conjunction with user terminal 200 and/or medical information providing device 300.

To this end, service server 100 can include at least one artificial intelligence model. At this time, the artificial intelligence model can be configured to extract spatial structural features and temporal motion features of the myocardium from a single echocardiographic image, either individually or in combination, and to calculate the presence of left ventricular outflow tract obstruction (binary classification) or the degree of left ventricular outflow tract obstruction (continuous value/score) based on the extracted features. In this case, the training data can be obtained from the hospital PACS, electronic medical record (EMR), research database, etc., and the predictive performance of the model can be maintained and improved through periodic updates.

In addition, the service server 100 compares the prediction results with actual clinical data to calculate a prediction index (Deep Learning-derived LVOT obstruction index, DLI-LVOTO), and stores it in a database to be used for long-term model validation and performance assessment. Furthermore, the service server 100 can combine the prediction results with clinical information such as age, sex, heart rate, and drug administration histories of the patient to calculate a prognosis grade or risk level for each patient, and transmit the result to the user terminal 200 in real time to support decision making of the medical staff.

Meanwhile, service server 100 can improve the prediction accuracy of the artificial intelligence model by performing periodic training updates, and maintain the reliability of the prediction model by continuously collecting and correcting new clinical data. Such a service server 100 can be implemented as any one of a cloud server, a web server for processing medical data, or a local hospital server, and can assist medical staff to rapidly assess patient prognosis and establish a treatment plan.

In addition, service server 100 can calculate performance assessment indicators such as accuracy, sensitivity, specificity, F1 score, and AUC for the output result of the artificial intelligence model. Through this, the reliability of the results of the left ventricular outflow tract obstruction prediction and prognostic assessment can be verified, and re-training can be performed by securing additional clinical data or supplementing the model structure if it falls short of the criteria. In this case, the feedback information collected from user terminal 200, whether the prediction result is hit, the medical staff assessment, and the follow-up echocardiography result can be used as the re-training data. Therefore, service server 100 can ensure long-term stability and clinical reliability of the artificial intelligence model through continuous performance assessment and periodic update, and can gradually improve the accuracy and the explainability of patient-specific prognostic assessment information.

Meanwhile, user terminal 200 is a generic term for a terminal possessed or used by a user who intends to use an information provision service provided by service server 100, and can be at least one terminal. Here, the user can include various users such as the patient himself/herself, medical staff, researchers, or administrators, but is not limited thereto.

User terminal 200 can upload the echocardiographic image to the service server 100, and receive and display the prediction result, prognostic assessment information, and related indicators provided from the server. To this end, user terminal 200 can be driven in the form of a mobile application, a web application, or a dedicated software, and can visualize and provide the result as a probability value, a rating indication, a score gauge, a warning message, a trend graph, or the like. In addition, user terminal 200 collects the clinical confirmation, the tracking test result, or the user feedback of the prediction result and transmits it to service server 100 so that it can be used for model performance check and re-training.

User terminal 200 may be a computer, UMPC (Ultra Mobile PC), workstation, net-book, Personal Digital Assistant(PDA), portable computer, web tablet, wireless phone, mobile phone, smart phone, tablet PC, smart watch, wearable terminal, e-book, portable multimedia player(PMP), portable game console, navigation device, black box or digital camera, other mobile communication terminal, etc., on which each user can install and execute multiple application programs (i.e., applications) desired, and is not limited to the terminals mentioned above.

Meanwhile, medical information providing apparatus 300 stores and manages an echocardiographic image, a clinical record, and a test history of at least one patient, and provides medical information required for service server 100. The medical information providing apparatus 300 can be implemented as an in-hospital EMR system, a medical image storage and transmission system (PACS), a cloud-based medical database, or an external medical data platform associated therewith. Further, the medical information providing apparatus 300 can update and store the prediction results and prognostic assessment information generated from the service server 100 to maintain consistency of clinical records.

Meanwhile, information provision system 1000 is not limited to the configuration shown in FIG. 1, and may also be configured to include other devices (terminals, servers, etc.) or may be implemented by excluding and integrating some configurations.

Through this information provision system 1000, users can receive comprehensive early identification of the risk of left ventricular outflow tract obstruction and prognostic assessment for hypertrophic cardiomyopathy patients, and based on this, practical medical effects such as rapid clinical decision making, patient-tailored management, and improved follow-up monitoring efficiency can be obtained.

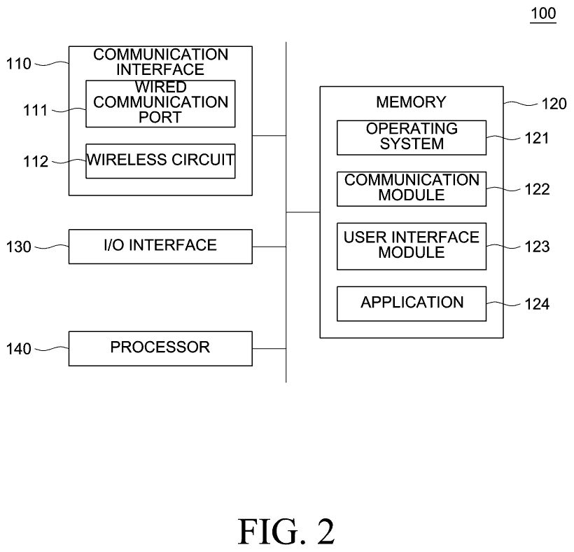

FIG. 2 is a block diagram showing the configuration of a service server that performs the task of providing information for the prediction of left ventricular outflow tract obstruction in hypertrophic cardiomyopathy according to an embodiment of the present disclosure.

Referring to FIG. 2, service server 100 can include a communication interface 110, a memory 120, an I/O interface 130, and a processor 140, and each component can communicate with each other through one or more communication buses or signal lines.

Communication interface 110 can be connected to user terminal 200 and the medical information providing device 300, as well as other devices, to exchange data through a wired/wireless communication network.

Meanwhile, a communication interface 110 enabling transmission and reception of such data includes a wired communication port 111 and a wireless circuit 112, wherein wired communication port 111 can include one or more wired interfaces, for example, Ethernet, a universal serial bus (USB), a Firewire, and the like. In addition, wireless circuit 112 can transmit and receive data to and from an external device through an RF signal or an optical signal. In addition, wireless communication can use at least one of a plurality of communication standards, protocols and technologies, such as GSM, EDGE, CDMA, TDMA, Bluetooth, Wi-Fi, VoIP, Wi-MAX, or any other suitable communication protocol.

Memory 120 can store data on at least one process (algorithm) for providing an information provision service or a program reproducing the process. In addition, memory 120 can further store processes for performing other operations, and is not limited thereto.

Meanwhile, memory 120 can build and store at least one pre-trained artificial intelligence-based model as needed as well as various data used in service server 100.

In various embodiments, memory 120 can include a volatile or nonvolatile recording medium capable of storing various data, commands, and information. For example, the memory 120 can include at least one type of storage medium among flash memory type, hard disk type, multimedia card micro type, card type memory (for example, SD or XD memory, etc.), RAM, SRAM, ROM, EEPROM, PROM, network storage, cloud, and blockchain database.

In various embodiments, memory 120 can store the configuration of at least one of operating system 121, communication module 122, user interface module 123, and one or more applications 124.

Operating system 121 (e.g., a built-in operating system such as LINUX, UNIX, MAC OS, WINDOWS, VxWorks, etc.) can include various software components and drivers for controlling and managing general system tasks (e.g., memory management, storage device control, power management, etc.), and can support communication between various hardware, firmware, and software components.

Communication module 122 can support communication with other devices through communication interface 110. Communication module 122 can include various software components for processing data received by wired communication port 111 of communication interface 110 or wireless circuit 112.

User interface module 123 can receive a user's request or input from a keyboard, a touch screen, a microphone, or the like through I/O interface 130 and provide a user interface on a display.

Application 124 can include programs or modules configured to be executed by one or more processors 140.

I/O interface 130 can connect an input/output device (not shown) of service server 100, e.g., at least one of a display, a keyboard, a touch screen, and a microphone, to the user interface module 123. I/O interface 130 can receive a user input (e.g., voice input, keyboard input, touch input, etc.) together with user interface module 123 and process a command according to the received user input.

Processor 140 can be connected to communication interface 110, memory 120, and I/O interface 130 to control the overall operation of service server 100 and perform various commands through an application or a program stored in memory 120.

Processor 140 can correspond to an operation device such as a central processing unit (CPU) or an application processor (AP). In addition, processor 140 can be implemented in the form of an integrated chip (IC) such as a system on chip (SoC) in which various computing devices are integrated. Alternatively, the processor 140 can include a module for calculating an artificial neural network model, such as a neural processing unit (NPU).

Specifically, processor 140 can be configured to acquire echocardiographic images of specific patients in response to a user's request to provide a service, perform pre-processing, input the pre-processed echocardiographic images into a pre-trained artificial intelligence model, and then, when a prediction result on whether or not there is left ventricular outflow tract obstruction (LVOTO) is output from the artificial intelligence model, generate prognostic assessment information based on the result.

In this case, in performing the preprocessing operation, processor 140 can perform at least one of noise removal, luminance correction, contrast correction, image resolution normalization, region of interest (ROI) extraction, time interval division, or frame-to-frame alignment in the echocardiographic image. However, these are only examples and are not limited to them.

For example, processor 140 normalizes the frame resolution of the input echocardiographic image, detects regions of interest of the left ventricle and the ventricular septum to remove background noise, and divides the time interval into regular intervals to perform alignment between frames, thereby converting the image into a form that can be processed by an artificial intelligence model. This preprocessing process is to clarify the myocardial boundary and contraction pattern, and to minimize noise due to differences in imaging conditions.

Meanwhile, processor 140 can drive an artificial intelligence model including a first neural network for extracting spatial structural features and a second neural network for extracting temporal motion features. That is, the artificial intelligence model is configured to extract spatial structural characteristics and temporal motion characteristics of the myocardium from the preprocessed echocardiographic image, either individually or in combination. For example, the first neural network is implemented as a three-dimensional convolutional neural network (3D-CNN), and the second neural network can be a two-dimensional convolutional neural network (2D-CNN) or a transformer-based network that analyzes changes in the time axis of the myocardium using automatically generated M-mode representations.

Further, processor 140 can automatically track the myocardial motion trajectory in the echocardiographic image through the second neural network to create a multi-level M-mode feature map, and embed it, thereby improving the precision of prediction regarding left ventricular outflow tract obstruction. Furthermore, the output embedding result of the second neural network can be combined with the output of the first neural network to comprehensively determine whether there is left ventricular outflow tract obstruction, and the result can be output as a predicted result.

This prediction result can be a binary classification result that classifies the presence of left ventricular outflow tract obstruction, or can include a continuous value (score) that quantitatively predicts a pressure gradient of a left ventricular outflow tract. Processor 140 can calculate a predictive indicator (DLI-LVOTO) that quantifies the degree of left ventricular outflow tract obstruction based on these predictive results, which predictive indicator can be calculated based on at least one of contraction speed of the myocardium, travel distance, or timing in the echocardiographic image. Therefore, predictive indicators can be used to verify the quantitative predictive performance of the model through correlation with actual clinical pressure gradient (Peak PG).

Furthermore, processor 140 can be configured to generate prognostic assessment information by combining the prediction result output from the artificial intelligence model with clinical information of the patient (e.g., age, sex, heart rate, drug administration histories, etc.). In this case, the prognostic assessment information can include a prognosis grade or risk level calculated by comprehensively analyzing the prediction result and clinical data for each patient, and the treatment response prediction result can be calculated according to the prognosis grade or risk level. Accordingly, the medical staff can determine the treatment direction based on the prognostic assessment information or select patients in the risk group early.

Meanwhile, processor 140 can calculate performance assessment indicators, such as accuracy, sensitivity, specificity, F1 score, and AUC, for the output result of the artificial intelligence model, and compare them with reference values to determine the reliability of the prediction result. If it falls short of the criteria, processor 140 can control to re-train the corresponding model by securing additional clinical data, adjusting model parameters, or supplementing the structure. In this process, feedback information collected from a user terminal 200, whether a prediction result is hit, an assessment by a medical staff, or a follow-up echocardiography result can be utilized as re-training data, thereby continuously improving the long-term stability and clinical reliability of the artificial intelligence model.

As a result, processor 140 can be configured to integrally manage the entire pipeline leading to pre-processing of echocardiographic images, extraction of spatial and temporal features, prediction of whether there is left ventricular outflow tract obstruction, generation of prognostic assessment information, and control of performance assessment and retraining. Accordingly, information provision system 1000 according to the present disclosure can quickly and consistently assess the possibility and prognosis of left ventricular outflow tract obstruction in patients with hypertrophic cardiomyopathy with only a single echocardiographic image, and can significantly improve the diagnostic efficiency of medical staff and the accuracy of treatment plan establishment.

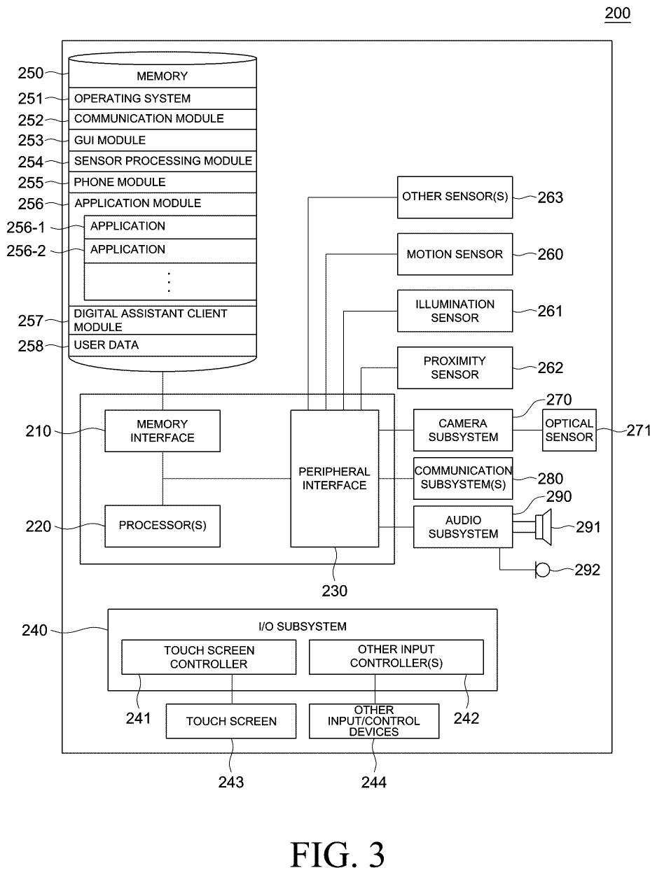

FIG. 3 is a block diagram illustrating a configuration of a user terminal receiving prognostic assessment information according to an embodiment of the present disclosure.

Referring to FIG. 3, user terminal 200 can include a memory interface 210, one or more processors 220, and a peripheral interface 230. Various components in user terminal 200 can be connected by one or more communication buses or signal lines.

Memory interface 210 can be connected to memory 250 to transmit various data to processor 220. Here, memory 250 can include at least one type of storage medium among flash memory type, hard disk type, multimedia card micro type, card type memory (for example, SD or XD memory, etc.), RAM, SRAM, ROM, EEPROM, PROM, network storage, cloud storage, or blockchain database.

In various embodiments, memory 250 can store a web/app application or program built to provide an information provision service by service server 100. In addition, the memory 250 can store various types of information about the user, and can store various types of information acquired through an application or a program.

In various embodiments, memory 250 can store at least one of an operating system 251, a communication module 252, a graphical user interface (GUI) module 253, a sensor processing module 254, a telephone module 255, and an application module 256. Specifically, operating system 251 can include instructions for processing a basic system service and instructions for performing hardware tasks. Communication module 252 can communicate with at least one of another device, computer, and server. Graphic user interface (GUI) module 253 can process a graphic user interface. Sensor processing module 254 can process sensor-related functions (e.g., process voice input received through one or more microphones 292). Phone module 255 can process phone-related functions. Application module 256 can perform various functions of a user application, such as electronic messaging, web browsing, media processing, browsing, imaging, and other process functions. In addition, user terminal 200 can store one or more software applications 256-1, 256-2 (e.g., a service application) associated with any one type of service in memory 250.

In various embodiments, memory 250 can store a digital assistant client module 257 (hereinafter, referred to as a DA client module) for providing prognostic assessment information, and accordingly store commands for performing client-side functions of the digital assistant (e.g., commands for performing a prognostic assessment information providing function) and various user data 258 (e.g., other data such as user-customized vocabulary data, preference data, the user's electronic address book, etc.).

Meanwhile, DA client module 257 can obtain a user's voice input, text input, touch input, and/or gesture input through various user interfaces (e.g., I/O subsystem 240) provided in user terminal 200.

In addition, DA client module 257 can output audio-visual and tactile types of data. For example, DA client module 257 can output data composed of a combination of at least one of voice, sound, notification, text message, menu, graphic, video, animation, and vibration. In addition, DA client module 257 can communicate with a digital assistant server (not shown) using communication subsystem 280.

In various embodiments, DA client module 257 can collect additional information about the surrounding environment of user terminal 200 from various sensors, subsystems, and peripheral devices to configure a context associated with the user input. For example, DA client module 257 can infer a user's intention by providing context information together with a user input to the digital assistant server. Here, the situation information that can be accompanied by a user input can include sensor information, for example, lighting, ambient noise, ambient temperature, images of the surrounding environment, video, and the like. For another example, the context information can include a physical state (e.g., device orientation, device location, device temperature, power level, speed, acceleration, motion pattern, cellular signal strength, etc.) of user terminal 200. As another example, the context information can include information related to a software state of user terminal 200 (e.g., a process being executed by user terminal 200, an installed program, past and current network activity, background service, error log, resource use, etc.).

In various embodiments, memory 250 can include additional or deleted instructions. Furthermore, user terminal 200 can also include an additional configuration in addition to the configuration shown in FIG. 3, or can exclude some configurations.

Processor 220 can control the overall operation of user terminal 200 and can execute various commands for using an information provision service provided by the service server 100 by driving an application or a program stored in memory 250.

Processor 220 can correspond to an operation device such as a central processing unit (CPU) or an application processor (AP). In addition, processor 220 can be implemented in the form of an integrated chip (IC) such as a System on Chip (SoC) in which various computing devices for performing machine training, such as a Neural Processing Unit (NPU), are integrated.

In various embodiments, processor 220 can display various information and/or data for predicting left ventricular outflow tract obstruction in hypertrophic cardiomyopathy based on the platform through the user interface screen.

Peripheral interface 230 can be connected to various sensors, subsystems, and peripheral devices to provide data so that user terminal 200 can perform various functions. Here, it can be understood that a function performed by user terminal 200 is performed by processor 220.

Peripheral interface 230 can obtain data from motion sensor 260, illumination sensor (optical sensor) 261, and proximity sensor 262 so that user terminal 200 can perform orientation, light, and proximity sensing functions. As another example, peripheral interface 230 can obtain data from other sensors 263 (a positioning system-GPS receiver, a temperature sensor, and a biometric sensor), through which user terminal 200 can perform functions related to other sensors 263.

In various embodiments, user terminal 200 can include a camera subsystem 270 connected with peripheral interface 230 and an optical sensor 271 connected therewith, such that user terminal 200 can perform functions such as inquiry of echocardiographic images or uploading of reference images.

In various embodiments, user terminal 200 can include a communication sub-system 280 connected to peripheral interface 230. Communication subsystem 280 consists of one or more wired/wireless networks and can include various communication ports, radio frequency transceivers, and optical transceivers.

In various embodiments, a user terminal 200 includes an audio subsystem 290 connected with a peripheral interface 230, and such an audio subsystem 290 includes one or more speakers 291 and one or more microphones 292, such that user terminal 200 can perform voice-operated functions, such as voice recognition, voice reproduction, digital recording, and telephone functions.

In various embodiments, user terminal 200 can include an I/O subsystem 240 connected to peripheral interface 230. For example, I/O subsystem 240 can control touch screen 243 included in the user terminal 200 through touch screen controller 241.

For example, touch screen controller 241 can detect a user's contact and movement or cessation of contact and movement using any one of a plurality of touch sensing technologies, such as capacitive, resistive, infrared, surface acoustic wave technology, proximity sensor array, etc. As another example, I/O subsystem 240 can control other input/control devices 244 included in user terminal 200 through other input controller(s) 242. As an example, the other input controller(s) 242 can control pointer devices such as one or more buttons, a rocker switch, a thumb-wheel, an infrared port, a USB port, and a stylus.

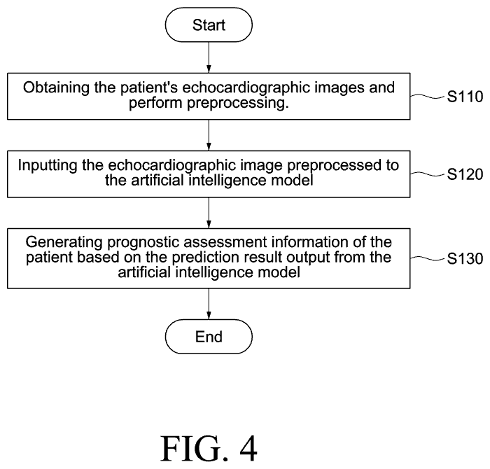

FIG. 4 is a flowchart schematically illustrating a method for providing information for the prediction of left ventricular outflow tract obstruction in hypertrophic cardiomyopathy according to an embodiment of the present disclosure.

Referring to FIG. 4, processor 140 acquires an echocardiographic image of a patient and performs pre-processing S110.

In this step S110, processor 140 can receive the echocardiographic image of the patient from user terminal 200 or medical information providing apparatus 300, and perform preprocessing of at least one of noise cancellation, luminance correction, contrast correction, image resolution normalization, region of interest extraction, time interval division, or frame-to-frame alignment on this echocardiographic image.

This allows the left ventricular outflow tract and the hypertrophic areas in the echocardiographic image to be clearly distinguished, allowing data to be converted into a form that can be stably processed by the artificial intelligence model. Here, the echocardiographic image can generally be a B-mode image based on the brightness of the tissue, but is not limited thereto.

Next, the processor 140 inputs the echocardiographic image preprocessed by step S110 to artificial intelligence model S120.

In this case, the artificial intelligence model can include a convolutional neural network (CNN)-based image analysis network and a recurrent neural network (RNN) or transformer-based time series analysis module.

In this step S120, processor 140 predicts through this artificial intelligence model at least one of whether there is left ventricular outflow tract obstruction, the pressure gradient (Peak PG), or the degree of left ventricular outflow tract obstruction, and calculates the prediction result in the form of a numerical indicator (DLI-LVOTO, etc.). Here, the output of the artificial intelligence model, that is, the prediction result, can include not only a simple normal/abnormal determination, but also a quantitative assessment result such as a change trend of the corresponding indicator, prediction reliability, and risk level.

Next, processor 140 generates prognostic assessment information of the patient based on the prediction result output from artificial intelligence model S130.

In this step S130, processor 140 can comprehensively analyze at least one of the predicted DLI-LVOTO value, the pressure gradient, or the degree of myocardial hypertrophy to calculate the possibility of future deterioration of the patient's condition, whether surgery or drug treatment is necessary, and treatment response prediction information. Here, the prognostic assessment information can be expressed in the form of at least one of a graph, a numerical index or a grade (e.g., a qualitative grade such as good prognosis/attention/worse risk of deterioration), and the prognostic assessment information is transmitted to user terminal 200 so that the medical staff can immediately check it.

Meanwhile, although not shown in FIG. 4, according to various embodiments of the present disclosure, processor 140 calculates performance assessment indicators such as accuracy, sensitivity, specificity, F1 score, and AUC for the prediction result of the artificial intelligence model, and if it falls short of the standard, additional clinical data can be obtained, or re-training can be performed by adjusting the model parameter or supplementing the structure. In this process, medical staff feedback provided through user terminal 200, whether a prediction result is hit, a tracking test result, and the like can be used as re-training data.

Accordingly, processor 140 does not stop at simply receiving the echocardiographic images and outputting the prediction results, but also supports patient-specific treatment judgment by generating prognostic assessment information based on the prediction results, and can improve the reliability and clinical utility of the prediction through continuous performance update.

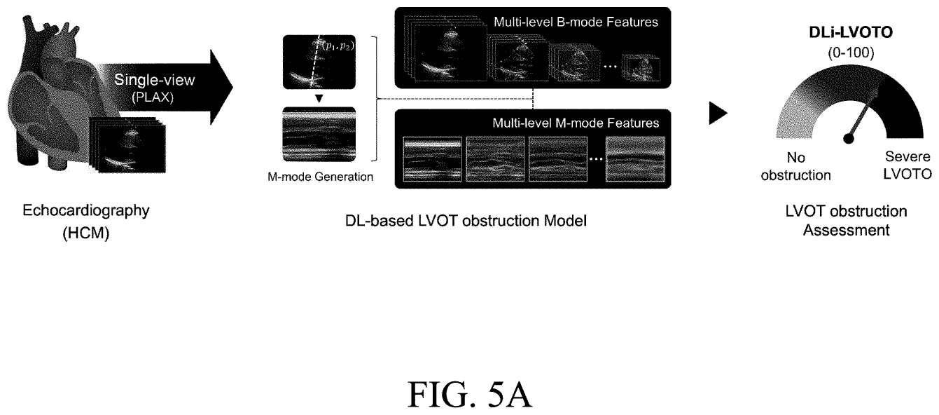

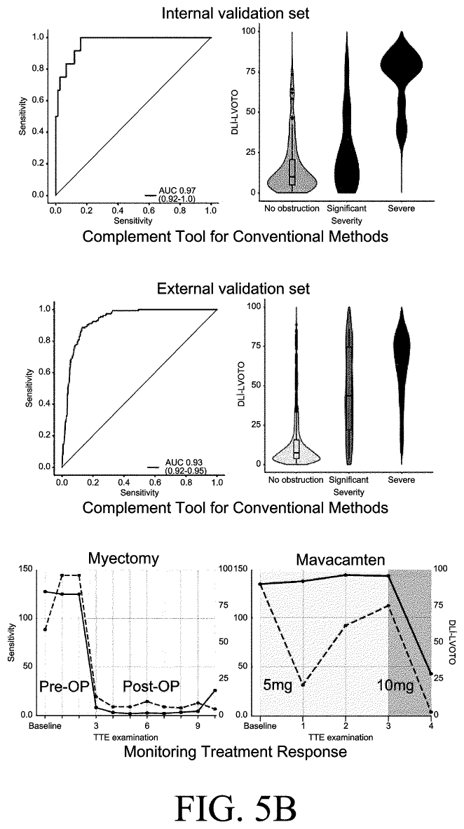

FIGS. 5A and 5B are diagrams schematically illustrating a pipeline for predicting whether there is left ventricular outflow tract obstruction based on a single echocardiographic image using an artificial intelligence model, according to an embodiment of the present disclosure.

Referring to FIG. 5A, when a patient's echocardiographic image (Parasternal Long-Axis View, PLAX) is input, processor 140 generates an M-mode image based on the corresponding echocardiographic image, that is, a B-mode image, and extracts a spatial structure feature and a temporal motion feature of a myocardium from the B-mode image and the M-mode image at multiple levels (multi-level).

Specifically, processor 140 analyzes spatial structural features such as shape and thickness of the myocardium and opening and closing of the left ventricular outflow tract from the B-mode image using a three-dimensional convolutional neural network (3D CNN), and at the same time analyzes changes in myocardial contraction and movement patterns according to the time axis from the M-mode image using a two-dimensional convolutional neural network (2D CNN). These two features are embedded and integrated, and are used as input to an artificial intelligence model that predicts the presence and extent of left ventricular outflow tract obstruction.

Processor 140 calculates a predictive indicator (DLI-LVOTO) that quantifies the degree of left ventricular outflow tract obstruction, based on the predictive result of the artificial intelligence model. This predictive indicator is expressed as a continuous value ranging from 0 to 100, with higher values indicating a more severe degree of closure.

For example, as shown on the right side of FIG. 5A, when the DLI-LVOTO value is low, it can be assessed as a ‘No obstruction’, and when it is high, it can be assessed as a ‘Severe LVOTO’.

For example, this feature extraction and prediction process can be performed by a pre-trained deep learning-based artificial intelligence model. This artificial intelligence model can include, for example, a three-dimensional convolutional neural network (3D-CNN) that extracts spatial structural features by inputting a frame sequence of a B-mode image, and a two-dimensional convolutional neural network (2D-CNN) that analyzes temporal motion patterns of the myocardium using M-mode representations.

Furthermore, processor 140 can fuse multi-level feature embeddings output from these two neural networks to predict whether there is left ventricular outflow tract obstruction, and convert the prediction result into a prediction index (DLI-LVOTO) and use it for prognostic assessment.

However, the configuration shown in FIG. 5A is only an exemplary embodiment, and the artificial intelligence model can be replaced with another type of deep learning structure (RNN, Transformer, etc.) that trains the time series characteristics of myocardial motion.

Thereafter, as illustrated in FIG. 5B, processor 140 verifies the performance of the model with respect to an internal validation set and an external validation set, and calculates indicators such as an ROC curve (AUC), a specificity, and a sensitivity to assess accuracy.

In addition, processor 140 can compare and analyze patient data after myomectomy or drug treatment (Mavacamten) to verify the clinical validity of the predictive indicator, thereby verifying whether the predictive indicator reflects the actual treatment response change.

Accordingly, according to the configuration illustrated in FIGS. 5A and 5B, information provision system 1000 according to the present disclosure can not only quantitatively predict the degree of left ventricular outflow tract obstruction with only a single echocardiographic image input, but also continuously monitor changes in the patient's prognosis according to the course of treatment.

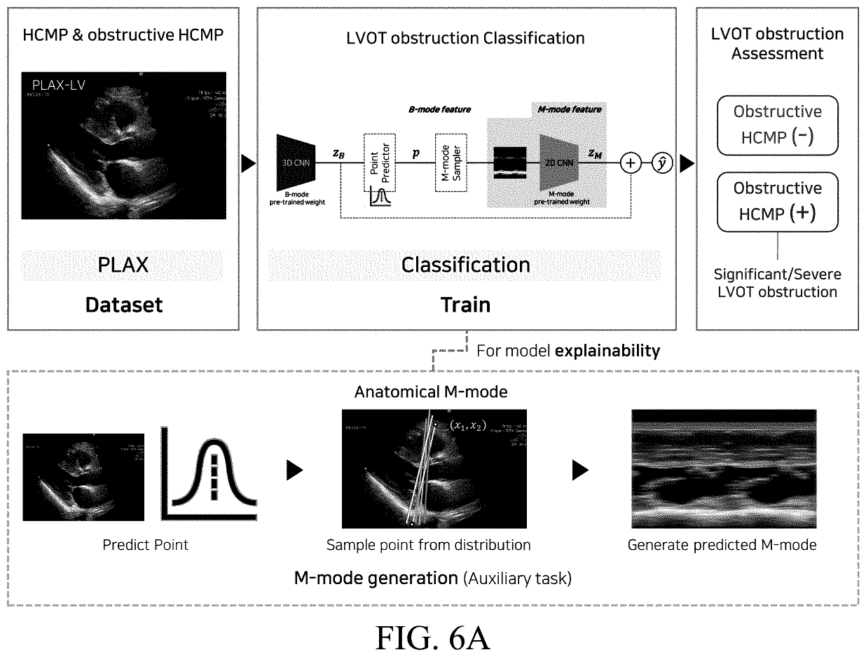

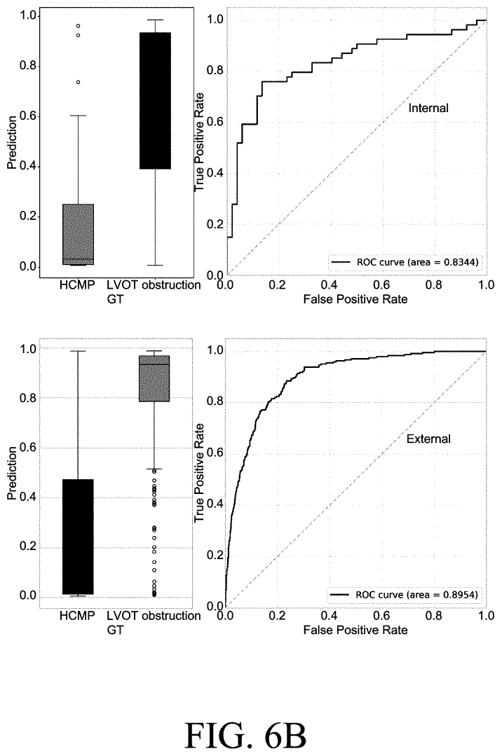

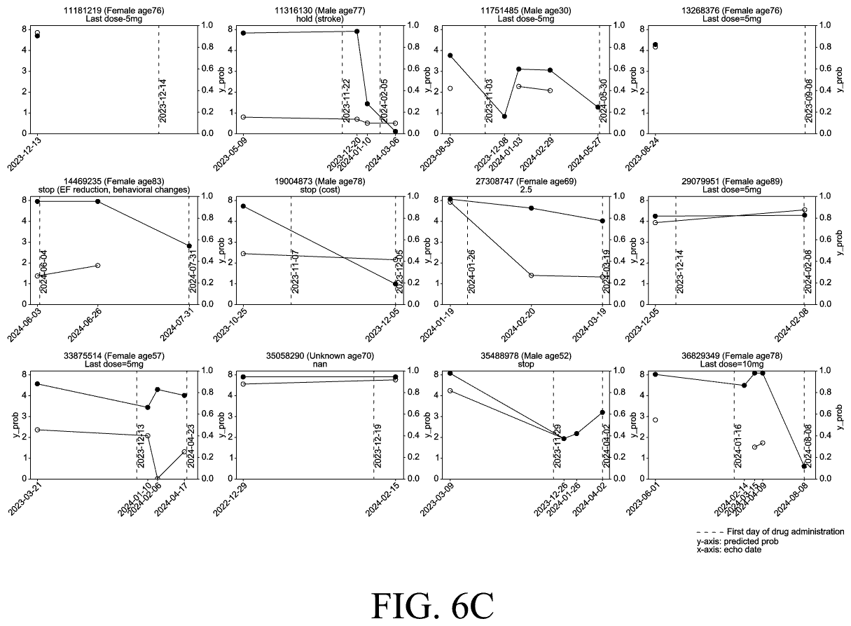

FIGS. 6A to 6C are diagrams schematically showing processes of training, performance evaluation, and drug response analysis of an artificial intelligence model for predicting left ventricular outflow tract obstruction in hypertrophic cardiomyopathy according to an embodiment of the present disclosure. Specifically, FIG. 6A illustrates a training data composition and classification pipeline, while FIGS. 6B and 6C illustrate a process of evaluating the performance of the artificial intelligence model and a process of analyzing drug response, using internal and external validation sets, respectively.

Referring to FIG. 6A, processor 140 collects echocardiographic images (PLAX) of patients with non-obstructive HCMP (Obstructive HCMP(−)) and obstructive HCMP (Obstructive HCMP(+)) to build a training dataset.

Processor 140 applies a three-dimensional convolutional neural network (3D CNN) to extract a feature (B-mode feature) from the B-mode image, which is an input echocardiographic image, and uses a point predictor to estimate a representative point of the left ventricular outflow tract area from the feature.

Subsequently, based on the predicted coordinates, the M-mode sampler automatically generates an M-mode image in the time axis direction, and the generated M-mode image extracts the temporal motion feature of the myocardium through a two-dimensional convolutional neural network (2D CNN).

Processor 140 integrates the B-mode feature (Z_{B}) and the M-mode feature (Z_{M}) to classify whether there is left ventricular outflow tract obstruction (LVOT obstruction), and divides the result into non-obstructive HCMP (Obstructive HCMP(−)) or obstructive HCMP (Obstructive HCMP (+)).

These classification results can be utilized to assess the severity (Significant/Severe LVOT obstruction) of left ventricular outflow tract, and the predicted point-based M-mode generation process can be used as an Auxiliary Task to improve the explainability of artificial intelligence models.

Referring to FIG. 6B, processor 140 assesses the performance of the artificial intelligence model for which training has been completed in FIG. 6A using an internal validation set and an external validation set.

In FIG. 6B, a box plot and a receiver operating characteristic (ROC) curve for prediction results are shown, respectively, and in the case of an internal validation set, an area under the curve (AUC) is about 0.8344 and in the case of an external set is about 0.8954, confirming that the artificial intelligence model of the present disclosure exhibits high prediction accuracy and generalization performance.

In addition, as shown in FIG. 6C, processor 140 analyzes a time series change in the predicted probability by inputting an echocardiographic image before and after drug administration in order to analyze the drug treatment effect (Mavacamten effect) of a patient with obstructive hypertrophic cardiomyopathy.

Each graph represents the echocardiographic date (echo date) on the x-axis and the predicted probability of the left ventricular outflow tract obstruction on the y-axis, and the dotted line represents the first administration date of the drug, and the black dot represents the model prediction probability.

For example, since the probability of left ventricular outflow tract obstruction tends to gradually decrease in most patients after drug administration, it can be confirmed that the artificial intelligence model of the present disclosure can quantitatively reflect the actual clinical treatment response.

Accordingly, as shown in FIGS. 6A to 6C, the artificial intelligence model of the present disclosure can accurately predict whether there is left ventricular outflow tract obstruction by comprehensively analyzing spatial and temporal features from a single echocardiographic image, and can also be effectively applied to analyzing clinical treatment effects.

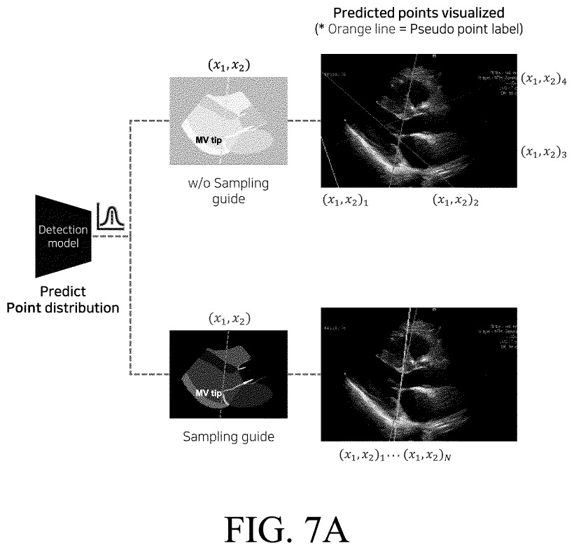

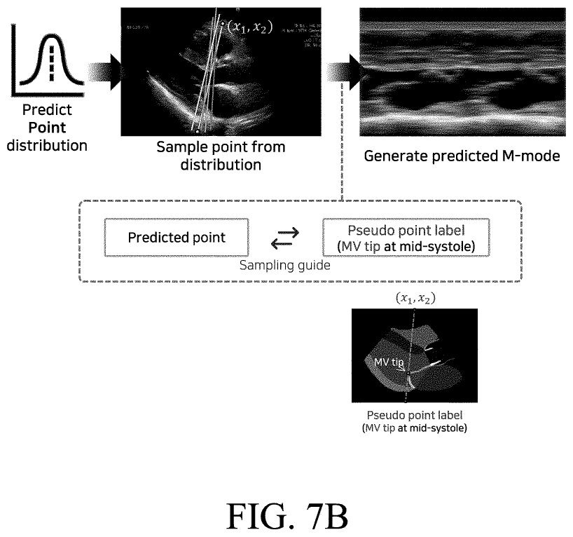

FIGS. 7A and 7B are diagrams illustrating examples of application of an M-mode generation process and a sampling guide based on a prediction point in an artificial intelligence model according to an embodiment of the present disclosure.

First, FIG. 7A illustrates a process in which an artificial intelligence model automatically predicts a position of a left ventricular outflow tract in an echocardiographic image, and automatically generates an M-mode image using the result.

Referring to FIG. 7A, processor 140 estimates a predicted point distribution indicating the position of the left ventricular outflow tract with respect to the input echocardiographic image. A sample point (x₁, x₂) is selected from this predicted distribution, and an M-mode sampling line is set based on the corresponding coordinates to generate a predicted M-mode image in the time-axis direction.

In this process, processor 140 can perform the training using a pseudo point label (MV tip at mid-systole) corresponding to the actual metrology point during the pre-training process, which acts as an inter-calibration relationship with the prediction point to form a sampling guide.

Meanwhile, FIG. 7B illustrates an example of performing prediction point visualization depending on whether a sampling guide is applied.

Referring to FIG. 7B, processor 140 can perform prediction point visualization depending on whether the sampling guide is applied. In the case where the sampling guide is not applied (w/o sampling guide), the prediction points (x₁, x₂) are irregularly distributed, but in the case where the sampling guide is applied (w/sampling guide), it can be seen that the prediction points corresponding to the actual mitral valve tip (MV tip) are aligned on the image.

As shown in the right image of FIG. 7B, it can be seen that each prediction point (x₁, x₂)₁ . . . (x₁, x₂)ₙ is distributed at a position consistent with the pseudo point label indicated by the orange line, and through this, the artificial intelligence model stably detects the anatomical reference point of the left ventricular outflow tract.

Accordingly, according to the configurations shown in FIGS. 7A and 7B, the artificial intelligence model of the present disclosure can predict the position of the left ventricular outflow tract from the single echocardiographic image stochastically and automate the generation of the time-axis M-mode image using the corresponding prediction point, thereby simultaneously improving the accuracy of myocardial motion pattern analysis and the explainability of the model.

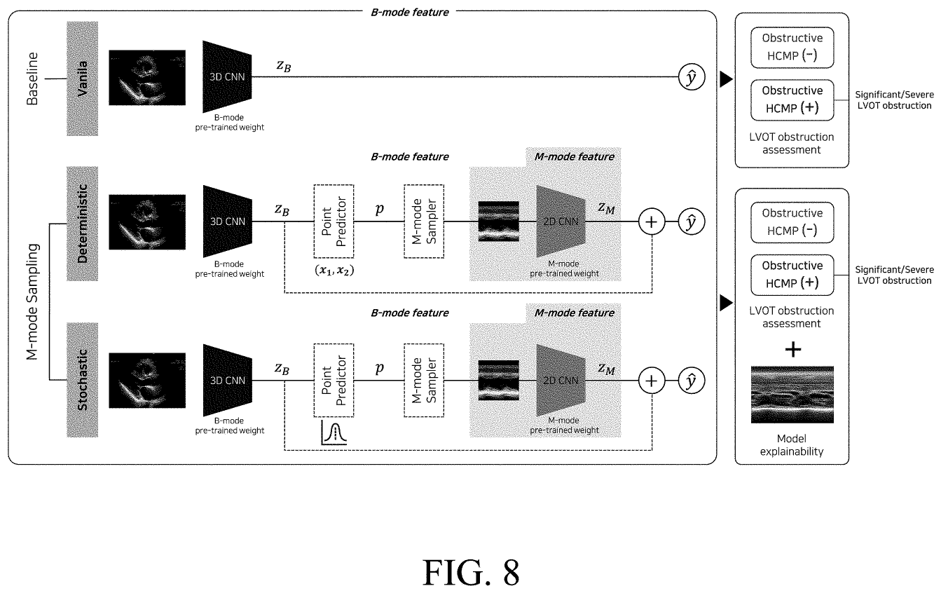

FIG. 8 is a diagram illustrating an artificial intelligence model structure for each M-mode sampling scheme according to an embodiment of the present disclosure.

Referring to FIG. 8, the artificial intelligence model of the present disclosure can be divided into three training structures each including a reference structure(baseline), a deterministic, and a stochastic M-mode sampling structure.

First, the reference model (Vanilla) uses the patient's echocardiographic image (B-mode image) as an input to extract spatial structural features (Z_{B}) through a three-dimensional convolutional neural network (3D CNN). In this case, the extracted feature (Z_{B}) is input to a classifier based on the pre-trained B-mode weights to predict whether there is left ventricular outflow tract obstruction (Obstructive HCMP), and the prediction result (ŷ) is output as one of “Obstructive HCMP (+)” or “Obstructive HCMP (−)”. Here, the reference model (Vanilla) is set as a baseline for experimental comparison and means a basic model to which an M-mode sampling structure or an auxiliary training module is not applied.

On the other hand, according to a deterministic M-mode sampling structure, a deterministic model estimates position coordinates (x₁, x₂) of a left ventricular outflow tract through a point predictor from a B-mode feature (Z_{B}) and generates an M-mode image in the time axis direction through an M-mode sampler based on this.

This generated M-mode image extracts temporal myocardial motion features (Z_{M}) by a two-dimensional convolutional neural network (2D CNN), and these two features (Z_{B}, Z_{M}) are combined to yield a final output (ŷ).

Therefore, since the deterministic model integrates and reflects spatial and temporal features through fixed coordinate-based sampling, it is possible to improve the precision of the prediction of left ventricular outflow tract obstruction.

Finally, according to a stochastic M-mode sampling structure, the probabilistic model is configured such that the point predictor calculates the probability distribution of predicted points instead of a single coordinate value.

Processor 140 stochastically extracts sample points (x₁, x₂) from this distribution, and generates an M-mode image based on the same.

Thereafter, the M-mode feature (Z_{M}) extracted through the two-dimensional convolutional neural network (2D CNN) is combined with the B-mode feature (Z_{B}) to produce a left ventricular outflow tract obstruction prediction result.

This probability-based sampling method can reflect uncertainty in predicting the position of the left ventricular outflow tract, resulting in more reliable results taking into account the fine variability of myocardial motion and improving the explainability of the model.

Accordingly, according to the configuration shown in FIG. 8, the artificial intelligence model of the present disclosure applies a deterministic or probabilistic M-mode sampling structure compared to the reference model using only the B-mode image, thereby providing an advantage of integrally reflecting spatial and temporal motion features, more accurately classifying whether there is left ventricular outflow tract obstruction and the severity, and visually explaining the internal inference process of the artificial intelligence model.

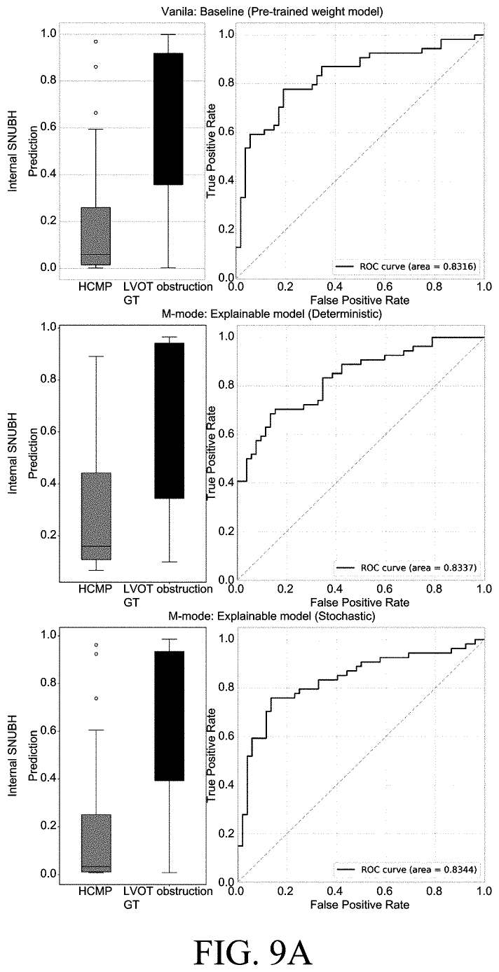

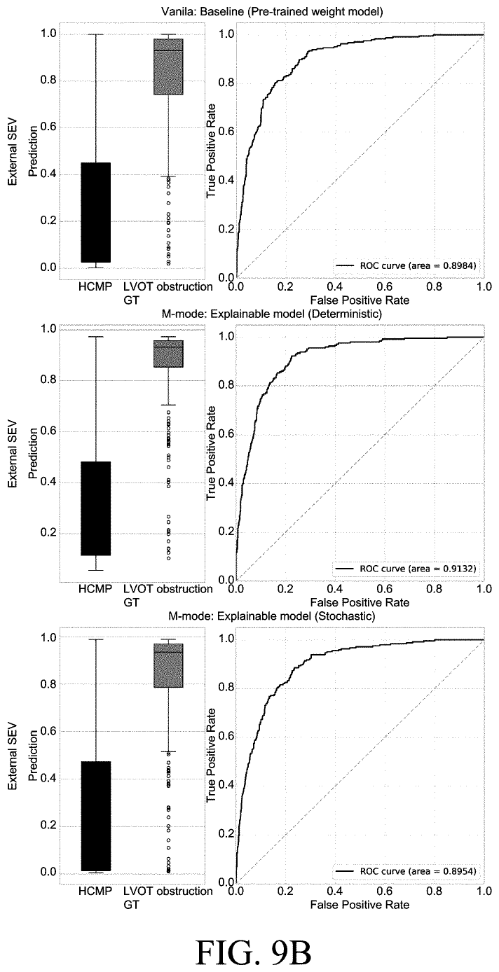

FIGS. 9A and 9B are views showing performance assessment results using internal and external validation datasets of an artificial intelligence model according to an embodiment of the present disclosure, FIG. 9A shows results for an internal validation set (SNUBH), and FIG. 9B shows results for an external validation set (SEV), respectively.

Referring to FIG. 9A and FIG. 9B respectively, three versions according to the structural differences of each model are compared and illustrated, and from the left, each of the results for the reference model (Vanilla: Pre-trained weight model), the deterministic M-mode based explainable model (hereinafter referred to as the ‘deterministic model’), and the stochastic M-mode based explainable model (hereinafter referred to as the ‘stochastic model’) are included.

For each model, the graph on the left is a box plot showing the distribution of predicted values compared to the actual ground truth (GT), and the graph on the right is a visualization of the classification performance (AUC) of the model through the ROC curve.

First, according to the internal validation result of FIG. 9A, the AUC of the reference model is about 0.8316, the deterministic model is about 0.8337, and the probabilistic model is about 0.8344, all showing similar or slightly improved prediction performance. This suggests that the M-mode sampling structure improves the explanatory power of the model while maintaining the prediction accuracy of the existing B-mode-based model.

In addition, according to the external validation result of FIG. 9B, the AUC of the reference model is about 0.8994, the deterministic model is about 0.9132, and the probabilistic model is about 0.8954, indicating that high generalization performance is secured for external data. In particular, the deterministic model shows the highest AUC in the external validation dataset, indicating that it is possible to reliably predict whether there is left ventricular outflow tract obstruction without relying on internal data training.

In addition, as shown in the box plot results of FIGS. 9A and 9B, the distribution of predicted values between the non-obstructive patient group and the obstructive patient group is clearly distinguished, which proves that the artificial intelligence model of the present disclosure can determine whether there is left ventricular outflow tract obstruction with high reliability.

Accordingly, according to the results shown in FIGS. 9A and 9B, the artificial intelligence model of the present disclosure has high classification accuracy in both internal and external validation environments, and in particular, it is shown that the explainability and clinical reliability of the model can be simultaneously improved through an M-mode-based structure.

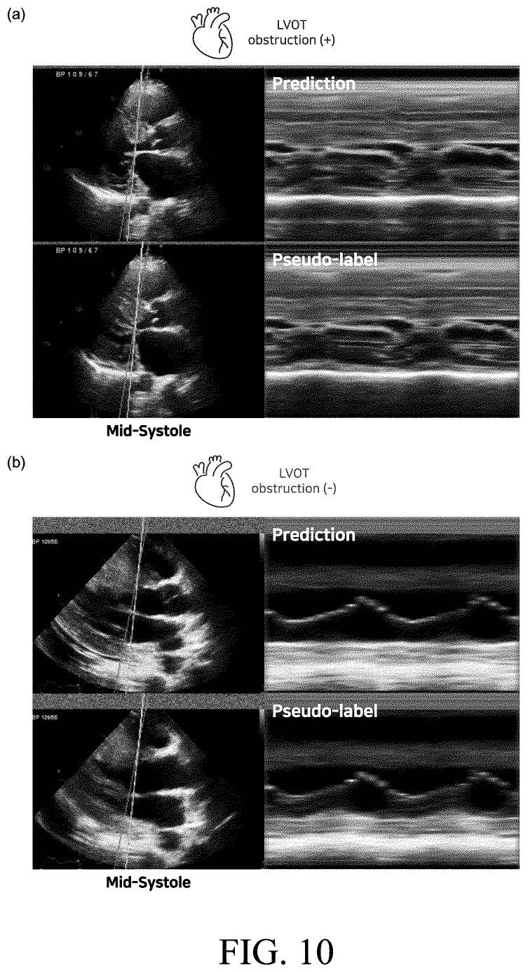

FIG. 10 is a diagram showing a visual comparison result according to whether there is left ventricular outflow tract obstruction predicted by an artificial intelligence model according to an embodiment of the present disclosure.
(a) of FIG. 10 is an example of an obstructive patient (LVOT obstruction (+)), and processor 140 performs M-mode sampling based on a prediction point in a mid-systole section of an echocardiographic image to calculate an M-mode image generated along a left ventricular outflow tract area.

In the predicted M-mode image shown at the top of (a), there is an aspect that the anterior wall of the myocardium and the mitral valve tip (MV tip) approach abnormally in the direction of the left ventricular septum, which is confirmed as a characteristic pattern reflecting the stenosis or closure of the left ventricular outflow tract.

Meanwhile, the pseudo-label shown at the bottom of (a) represents a reference coordinate defined based on the actual mitral valve tip (MV tip at mid-systole), and the visual correspondence of the model can be confirmed through the alignment with the prediction result. That is, the prediction point (blue line) and the pseudo label (orange line) almost coincide, indicating that the artificial intelligence model of the present disclosure accurately detects the actual anatomical reference point.
(b) of FIG. 10 is an example of a non-obstructive patient (LVOT obstruction(−)) and shows a comparison of a predicted M-mode image and a pseudo-label generated in the same mesenteric section.

In the predicted M-mode image shown at the top of (b), the movement of the mitral valve tip maintains a constant interval, and the change in opening and closing of the left ventricle outflow tract area is normally maintained. In addition, it can be confirmed that the alignment between the prediction point and the pseudo-label shown at the lower end of (b) is stably maintained, so that the model accurately reflects the normal myocardial motion pattern.

Accordingly, according to the results shown in FIG. 10, the artificial intelligence model of the present disclosure can automatically generate an M-mode image that accurately reflects the actual anatomical structure and motion pattern for both obstructive and non-obstructive patients with left ventricular outflow tract, thereby simultaneously improving the explainability and clinical reliability of the model.

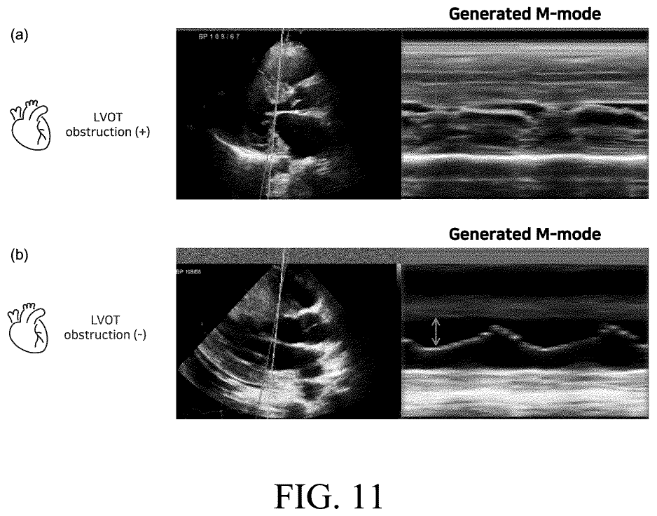

FIG. 11 is a diagram illustrating an example of interpretation of an M-mode image generated by an artificial intelligence model as a prediction point-based sampling guide according to an embodiment of the present disclosure.
(a) of FIG. 11 shows an example of an obstructive patient (LVOT obstruction (+)), and (b) of FIG. 11 shows an example of a non-obstructive patient (LVOT obstruction(−)), respectively, and in both cases, the left side represents an input B-mode (PLAX) image and a prediction/guide line, and the right side represents an M-mode image automatically generated along the line.

First, referring to (a) of FIG. 11, an M-mode image generated in a mid-systole section shows a pattern in which an anterior wall and a mitral valve tip (MV tip) structure abnormally approach or contact (SAM pattern) in a septum direction (red arrow).

As a result, the gap between the waveforms is narrowed or discontinuous high-echo bands are formed on the M-mode image, reflecting the characteristic findings accompanying left ventricular outflow tract stenosis or obstruction.

The artificial intelligence model of the present embodiment can analyze both quantitatively and qualitatively the closing pattern by automatically predicting a corresponding line from a single echocardiographic image and imaging it in a time axis direction.

Meanwhile, referring to (b) of FIG. 11, in the M-mode image generated by the same procedure, the interval between the mitral valve tip and the septum (E-septal deviation) is kept constant, and periodic and regular wavy waveforms appear (blue bidirectional arrows).

As a result, the SAM pattern or abnormal contraction pattern is not observed, suggesting that the opening and closing of the left ventricular outflow passage is normally maintained.

Accordingly, according to the results shown in FIG. 11, the artificial intelligence model of the present disclosure can visually explain the presence of left ventricular outflow tract obstruction through a consistent pipeline of prediction lines, M-mode generation, and pattern reading, and can simultaneously improve the interpretability and clinical reliability of the artificial intelligence model.

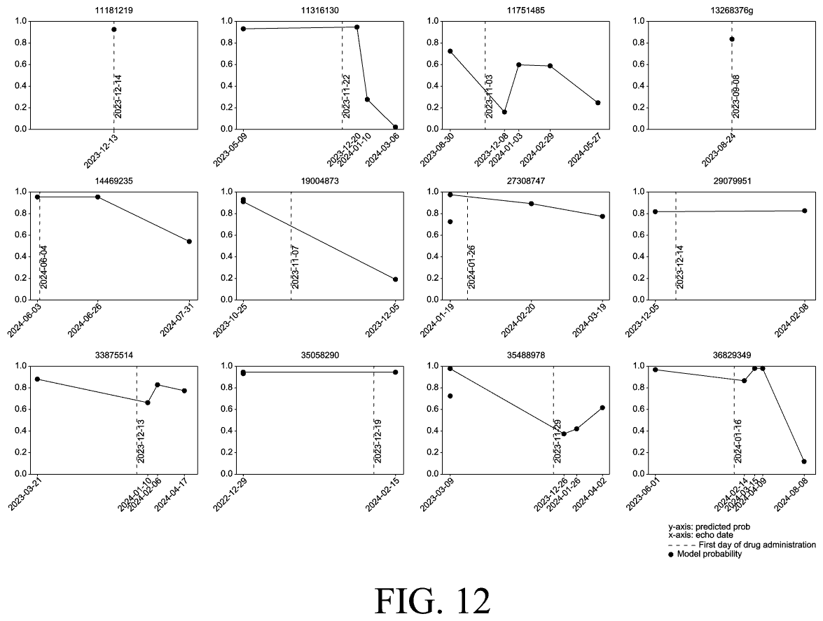

FIG. 12 is a view illustrating an example of tracking a Mavacamten treatment response to a patient with obstructive hypertrophic cardiomyopathy using an artificial intelligence model according to an embodiment of the present disclosure.

Each graph in FIG. 12 is based on the results of consecutive echocardiograms from different patients, with the x-axis representing the examination date (echo date) and the y-axis representing the predicted probability of left ventricular outflow tract obstruction. Here, the dotted line means the first administration time point of the drug, and each point represents the probability of closure predicted by the model.

As shown in FIG. 12, in most patients, the predicted probability after drug administration tends to gradually decrease, suggesting that the artificial intelligence model of the present disclosure can quantitatively reflect the effect of relieving left ventricular outflow tract obstruction according to actual drug treatment.

On the other hand, in some patients, the rate or trend of change in the predicted probability appears differently, and through these individual differences, clinical judgment such as prediction of treatment response and adjustment of customized drug dose can be supported.

Therefore, FIG. 12 shows that the artificial intelligence model of the present disclosure can be used as a clinical utilization tool capable of quantitatively and visually assessing changes in a patient's condition before and after treatment based on a single echocardiographic image input.

As described above, according to the present disclosure, by processing a single echocardiographic image as input through a unified pipeline—including preprocessing, determining whether there is left ventricular outflow tract obstruction, quantifying obstruction severity (calculating DLI-LVOTO), and predicting treatment response—, an AI-based service that provides information for assessing myocardial function and predicting left ventricular outflow tract obstruction that can be utilized in real clinical settings can be automatically provided. This can solve the problem of lack of objective indicators and inefficiency of treatment response tracking, which have been pointed out as limitations in the existing manual-based reading and qualitative assessment methods, and improve the accuracy of patient-specific prognosis prediction and treatment decision making, thereby securing clinical reliability and practical utilization at the same time.

Although the embodiments of the present disclosure have been described in more detail with reference to the attached drawings, the present disclosure is not necessarily limited to these embodiments, and various modifications can be made without departing from the technical idea of the present disclosure. Accordingly, the embodiments disclosed in the present disclosure are not intended to limit the technical idea of the present disclosure, but to explain it, and the scope of the technical idea of the present disclosure is not limited by these embodiments. Therefore, it should be understood that the embodiments described above are exemplary in all aspects and not restrictive. The protection scope of the present disclosure should be interpreted by the claims below, and all technical ideas within the equivalent scope should be interpreted as being included in the scope of the rights of the present disclosure.

What is claimed is:

1. A method for providing information for a prediction of a left ventricular outflow tract obstruction in a hypertrophic cardiomyopathy, performed by an apparatus, the method comprising:
obtaining an echocardiographic image of a patient and performing a preprocessing;
inputting a preprocessed echocardiographic image into a pre-trained artificial intelligence model to extract spatial structural features and temporal motion features of a myocardium, either individually or in combination, from a single echocardiographic image as input, and to predict whether there is left ventricular outflow tract obstruction based on the extracted spatial structural features and temporal motion features and output a prediction result; and
generating, when a prediction result based on the preprocessed echocardiographic image is output from the pre-trained artificial intelligence model, prognostic assessment information for the patient based on the prediction result.

2. The method of claim 1, wherein the performing the preprocessing performs: at least one of noise removal, luminance correction, contrast correction, image resolution normalization, region of interest (ROI) extraction, time interval division, or frame-to-frame alignment in the echocardiographic image.

3. The method of claim 1, wherein the pre-trained artificial intelligence model comprises: a first neural network for extracting spatial structural features from the preprocessed echocardiographic image and a second neural network for extracting temporal motion features from a frame sequence of the preprocessed echocardiographic image.

4. The method of claim 3, wherein the first neural network is implemented as a 3D convolutional neural network (3D-CNN), and the second neural network is a 2D convolutional neural network (2D-CNN) or a transformer-based network configured to analyze a change in a time axis of the myocardium using an automatically generated M-mode representation.

5. The method of claim 3, wherein the second neural network generates a multi-level M-mode feature map by automatically tracking a myocardial motion trajectory in the echocardiographic image, and embeds the multi-level M-mode feature map to improve an accuracy of prediction of left ventricular outflow tract obstruction.

6. The method of claim 5, wherein the pre-trained artificial intelligence model predicts whether there is the left ventricular outflow tract obstruction by combining an embedding result output from the second neural network with an output of the first neural network.

7. The method of claim 1, wherein the prediction result is a binary classification result for classifying presence of a left ventricular outflow tract obstruction, or includes at least one of continuous scores quantitatively predicted for a pressure gradient of the left ventricular outflow tract.

8. The method of claim 7, wherein the prediction result is calculated as a prediction index (DLI-LVOTO) obtained by quantifying a degree of the left ventricular outflow tract obstruction, and the prediction index is calculated based on at least one of a contraction speed of a myocardium, a movement distance, or a timing difference in the preprocessed echocardiographic images.

9. The method of claim 1, wherein the generating the prognostic assessment information includes:
combining the prediction result with at least one piece of clinical information about the patient to calculate a prognosis grade or risk level; and
outputting a treatment response prediction result according to the calculated prognosis grade or risk level.

10. An apparatus for providing information for a prediction of a left ventricular outflow tract obstruction in a hypertrophic cardiomyopathy, the apparatus comprising:
a communication interface;
a memory; and
a processor operatively connected with the communication interface and the memory, the processor configured to:
obtain an echocardiographic image of a patient and perform pre-processing;
extract spatial structural features and temporal motion features of a myocardium, either individually or in combination, using a single echocardiographic image as an input, to input the pre-processed echocardiographic image to an artificial intelligence model that is pre-trained to predict whether there is the left ventricular outflow tract obstruction based on the extracted features and to output a prediction result; and
generate prognostic assessment information for the patient based on the prediction result, when a prediction result based on the pre-processed echocardiographic image is output from the artificial intelligence model.
